(19) United States
(12) Patent Application Publication
(10) Pub. No.: US 2010/0233222 A1
(43) Pub. Date: Sep. 16, 2010

(54) COSMETIC SKIN TREATMENT PROCESS

(75) Inventors: Franck GIRIER DUFOURNIER, Paris (FR); Anne Liechty, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/787,055

(22) Filed: May 25, 2010

Related U.S. Application Data
(62) Division of application No. 10/902,894, filed on Aug. 2, 2004, now abandoned.
(60) Provisional application No. 60/497,557, filed on Aug. 26, 2003.

(30) Foreign Application Priority Data
Aug. 1, 2003 (FR) 03 09573

(51) Int. Cl. A61K 8/02; A61K 8/18; A61Q 3/02; A61Q 1/02; A61Q 19/00; A61K 8/25; A61K 8/29; A61K 8/27; A61K 8/73; A61K 8/81; A61K 8/26

(52) U.S. Cl. 424/401; 424/61; 424/63; 424/724; 424/683; 424/617; 424/642; 514/60; 424/78.03; 424/684

(57) ABSTRACT

Cosmetic treatment processes include application of cosmetic compositions to a skin surface by spraying, such compositions including at least one filler in a physiologically acceptable medium, the at least one filler giving the composition a haze index H of 40% or more and a hemispheric transmission factor Th of 70% or more.

### CROSS-REFERENCE TO RELATED APPLICATIONS

This is a Division of application Ser. No. 10/902,894 filed Aug. 2, 2004, which in turn claims the benefit of French Application No. 03 09573 filed on Aug. 1, 2003 and U.S. Provisional Application No. 60/497,557 filed on Aug. 26, 2003. The disclosure of the prior applications is hereby incorporated by reference herein in their entirety.

### BACKGROUND

The present invention relates to cosmetic treatment of the skin, mucous membranes and/or integuments, especially non-fibrous keratin materials.

More particularly, the invention is directed towards processes that are useful, for example, for at least partially masking skin defects.

It is common to wish to camouflage skin imperfections, in particular of the face or the neck, using foundations.

Conventional foundations generally provide coverage for large areas and are useful for matting the complexion and masking skin color imperfections, such as redness. However, these conventional foundations are not always suitable for also masking relief defects such as wrinkles.

To camouflage wrinkles and other relief defects, compositions containing “soft-focus” fillers are used. These compositions reduce the perception of the relief of the surface onto which they are applied.

The application of a composition containing soft-focus fillers, manually or using an applicator, onto a surface that has already been made up with a foundation leads to at least partial mixing of the two compositions. This intimate mixing of conventional foundation with compositions containing soft-focus fillers results in a reduction in the efficacy of such soft-focus compositions and detracts from the quality of the result.

There is thus a need for cosmetic skin treatment processes that make it possible especially to reduce the perception of relief defects, and that also makes it possible, where appropriate, to mask color defects and to make the complexion matte.

There is moreover a need to further improve the quality of makeup obtained using a compositions containing at least one soft-focus filler.

There is also a need to dissimulate defects without generating a mask effect on, and to maintain a natural appearance for, made-up skin.

### SUMMARY

According to embodiments of the present invention, cosmetic treatment processes, for example, makeup processes, for a surface of skin, mucous membranes or nails, are provided. In embodiments, exemplary processes comprise application of soft-focus cosmetic compositions onto surfaces. Exemplary soft-focus cosmetic compositions according to embodiments of the invention comprise, in a physiologically acceptable medium, at least one filler for giving compositions a haze index H of 40% or more and a hemispheric transmission factor Th of 70% or more. Soft-focus cosmetic compositions may be applied to surfaces, in embodiments, by spraying.

The term “spraying” denotes a distribution, preferably in the form of a mist, of more or less fine particles, for example, of droplets.

In embodiments, application of soft-focus cosmetic compositions by spraying can allow substantially uniform deposition thereof, which contributes towards even better camouflaging skin relief defects.

In embodiments, base compositions may be applied to a surface to be treated prior to application of soft-focus cosmetic compositions. Such base compositions may be, for example, colored, as in a conventional foundation.

Spraying advantageously makes it possible to deposit compositions in the form of thin films, for example of “veil” type, of soft-focus cosmetic compositions onto the surface of a base composition. Thus, any intimate mixing between the two compositions that might be harmful to the development of desired soft focus may be prevented.

Makeup methods according to embodiments of the invention may thus involve, in a first step, application by any conventional mode, for instance manually, using an applicator such as a brush or a sponge or by spraying, of a colored base cosmetic composition. A soft-focus cosmetic composition may then be superposed, in a second step, by spraying, onto this first makeup coat.

Embodiments of the invention may thus make it possible to obtain makeups that are satisfactory in terms of camouflage, whether of colored imperfections or of relief.

Application of soft-focus cosmetic compositions may also, in embodiments, be simultaneous with application of base compositions, for example, by means of using a spraying device comprising at least two nozzles.

Soft-focus cosmetic compositions may contain at least one coloring agent, for example, depending on whether compositions are intended to be superposed onto colored base compositions.

In embodiments, soft-focus cosmetic compositions are applied directly onto surfaces to be made up and comprise a coloring agent, the saturation C* of soft-focus cosmetic compositions may be between 17 and 60 or between 20 and 60.

A saturation value C* of greater than 17 allows a sufficient supply of color to be obtained, whereas a value of less than 60 makes it possible to not detract from the production of the soft focus and can also allow a natural makeup to be obtained.

For a better understanding of the invention as well as other aspects and further features thereof, reference is made to the following figure and descriptions.

### BRIEF DESCRIPTION OF THE DRAWINGS

FIG. 1 is a representation of hemispheric spectral transmission Th(λ) of a composition on a quartz slide.

FIG. 2 is a representation of direct spectral transmission Td(λ) of a composition on a quartz slide.

FIGS. 3 and 4 are schematic representations of exemplary spray devices that may be used in accordance with embodiments of the invention.

### DETAILED DESCRIPTION OF EMBODIMENTS

### Measurement of Hemispheric Transmission Factor Th and Calculation of Haze Index H

As indicated above, soft-focus cosmetic compositions according to embodiments of the invention may have a haze index H of 40% or more, 45% or more, 50% or more, 60% or more, 70% or more, 75% or more or 80% or more.

In embodiments, the hemispheric transmission factor Th of soft-focus cosmetic compositions may be 70% or more, 75% or more and 80% or more, or 85% or more.

The term “haze index H” denotes the magnitude ((Th−Td)/Th)·100, in which Th represents the hemispheric transmission factor and Td represents the direct transmission factor.

When the haze index H is high, compositions give a large soft focus, allowing defects to be masked by changing perception of relief.

The hemispheric transmission factor Th gives information regarding the transparency of compositions. That is, the higher the Th value, the more transparent the composition.

The Th and Td values may be measured using a spectrophotometer and an integration sphere, placed behind the composition that it is desired to characterize.

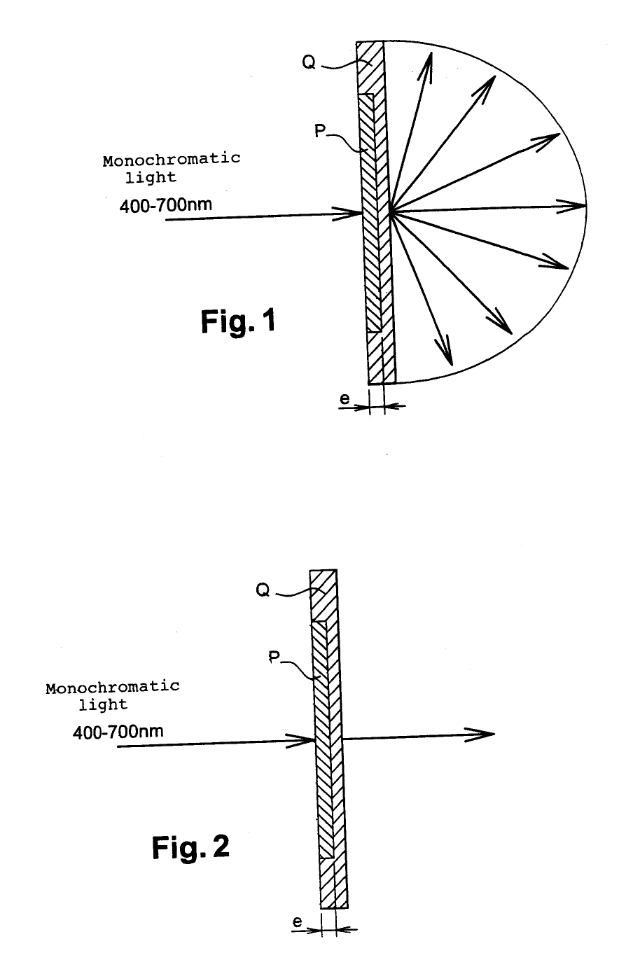

The hemispheric spectral transmission Th(λ) of a composition is defined by the ratio between the intensity of light of wavelength λ received by a sample of composition P and the intensity of the light released by this sample in all directions in a space delimited by a plane, as illustrated in FIG. 1.

The direct spectral transmission Td(λ) of a sample is defined by the ratio between the intensity of light of wavelength λ received by the sample and the intensity of the light released by this sample in the same direction of propagation as the incident light, as illustrated in FIG. 2.

The measurements of the hemispheric spectral transmission Th(λ) and direct spectral transmission Td(λ) may be performed according to the following experimental protocol.

The composition P to be analysed is spread onto a hollowed-out quartz slide Q, so as to form a layer with a thickness of 20 μm, and is then placed in an oven for five minutes at 37° C.

The Th(λ) value may be measured using, for example, a Varian Cary 300® spectrophotometer and a LABSPHERE® brand integration sphere placed behind the quartz slide containing the composition. The spectrophotometer is used in diffuse transmission mode and the wavelength λ of the monochromatic incident light ranges from 400 to 700 nm.

The measurement is performed in transmission mode % T, at a sweep speed of 240 nm/minute and in “double reverse” mode.

In a first stage, a calibration is performed by taking a first measurement on the empty quartz slide Q to obtain the maximum value of the transmitted intensity.

Next, the hemispheric spectral transmission Th(λ) is measured with the quartz slide containing the composition P to be analysed.

The Td(λ) value is measured using the same spectrophotometer, used in direct transmission mode and the wavelength λ of the incident monochromatic light ranges from 400 to 700 nm. The machine is set to transmission mode % T, at a sweep speed of 240 nm/minute, in “double” mode. An empty quartz slide is placed in a reference compartment and the quartz slide containing the composition is placed in a measuring compartment, and the Td(λ) is then measured.

The hemispheric transmission factor Th and direct transmission factor Td are calculated by establishing, respectively, the mean of all the Th(λ) or Td(λ) spectral values obtained when the wavelength ranges from 400 to 700 nm.

### Saturation C*

In embodiments, soft-focus cosmetic compositions are applied directly onto surfaces to be made up and comprise a coloring agent, the saturation C* of soft-focus cosmetic compositions are between 17 and 60 and between 20 and 60.

A saturation value C* of greater than 17 allows a sufficient supply of color to be obtained, whereas a value of less than 60 makes it possible to not detract from production of soft focus and can also allow natural makeup appearance to be obtained.

The saturation C* may be measured in the CIE L*a*b*C*h colorimetric space, in the following manner.

The composition whose saturation is to determined is placed up to the brim in a galvanized steel crucible having a minimum depth of 2.47 mm.

The crucible is then covered with a cover slide, such as, for example, 76×26 mm “LMR” H1 bevelled-edge cover slide from the company Labo-Moderne.

The saturation C* is measured using a spectrocolorimeter, for example a CM3700d spectrocolorimeter. The illumination and observation system of the spectrocalorimeter is set in d/8° reflectance mode, and the measurements are taken in exclusive specular reflection mode.

The UV emission is 100% inclusive.

The zoom position is medium (MAV); the aperture is medium; the configuration is CREIMM; the observation is 10° CIE 1964; and the illuminant is D65.

### Spraying Device

Embodiments of the invention include spraying devices comprising a container containing one or more soft-focus cosmetic compositions as defined above and discussed in detail below.

Spraying devices of various embodiments may comprise means for subjecting soft-focus cosmetic compositions to positive or negative pressure to allow spraying.

In embodiments, spraying of soft-focus cosmetic compositions may be performed by any means known to those skilled in the art, provided soft-focus properties are not affected.

In embodiments, spraying may take place, for example, using means for pressurizing soft-focus cosmetic compositions, for example, by subjecting such compositions to pressure from propellent gas.

Propellent gas may be packaged with soft-focus cosmetic compositions or separately, such as where such compositions are contained in a flexible bag inside a pressurized container.

Soft-focus cosmetic compositions, in embodiments, may also be pressurized by means of a pump, for example, a manually operated pump, which may or may not be with precompression.

In embodiments, soft-focus cosmetic compositions may also be sprayed by means of a Venturi effect in a spraying device of aerographic type.

Whatever spraying mode is adopted in embodiments, soft-focus cosmetic compositions may be sprayed via one or more spraying nozzles, with or without turbulence channels.

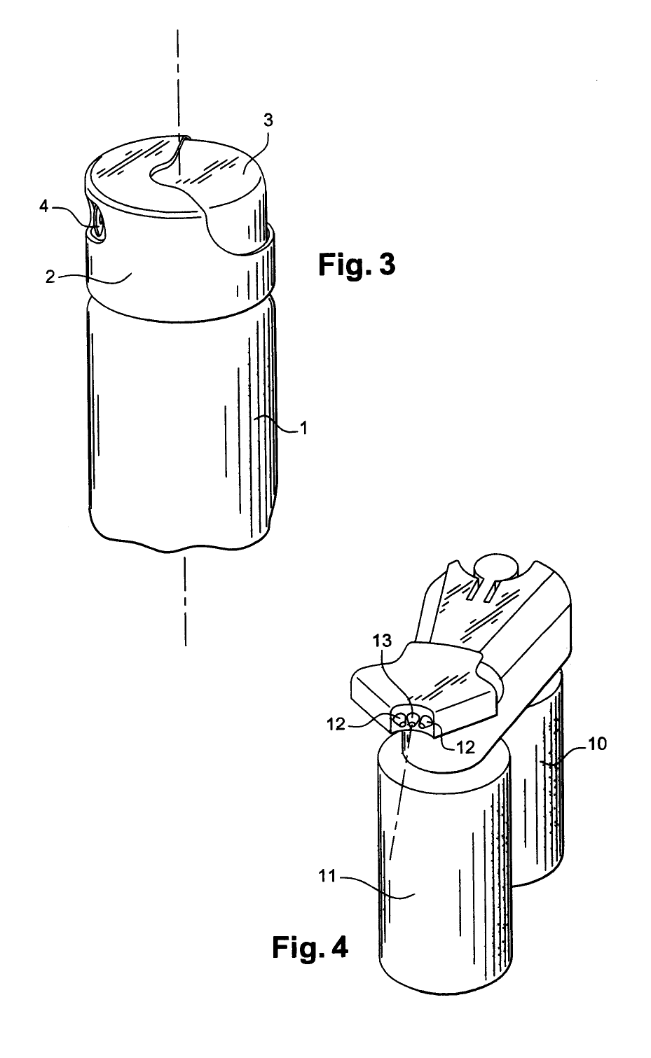

FIGS. 3 and 4 show two non-limiting examples of spraying devices that may be used in accordance with embodiments.

The device in FIG. 3 is, for example, of aerosol type and comprises pressurized container 1 containing soft-focus cosmetic compositions and propellent gas, distribution head 2 comprising push button 3 for actuating a valve, not shown, and spraying nozzle 4.

FIG. 4 shows another example of a spraying device of aerographic type, comprising first container 10 containing carrier gas and second container 11 containing soft-focus cosmetic compositions, carrier gas 10 being emitted, for example, via nozzles 12 upstream of a spraying nozzle 13. Negative pressure created by carrier gas flow causes suction in reservoir 11 of soft-focus cosmetic compositions.

Additional non-limiting examples of spraying devices that may be used in embodiments are described in WO 02/47618, which is incorporated herein by reference in its entirety.

According to embodiments, soft-focus cosmetic compositions are sprayed by means of propellent gas, for example, compressed gas such as compressed air or nitrogen, or liquefiable gas such as dimethyl ether, C₃₋₅ alkanes, for example propane, n-butane and isobutane, 1,1-difluoroethane and mixtures of dimethyl ether and of C₃₋₅ alkanes.

The weight ratio of soft-focus cosmetic compositions to propellent gas may range, in embodiments, for example, from 0.05 to 50 and from 1.5 to 25.

In embodiments, spraying may also be performed using spraying devices without carrier gas or propellent gas. Electromechanical or thermal devices are examples of such spraying devices.

### Soft-Focus Cosmetic Composition

### Soft-Focus Fillers

Soft-focus fillers that may be used in soft-focus cosmetic compositions in exemplary embodiments may comprise or consist of particles with a number-average size of 15 μM or less, 10 μm or less, 7.5 μm or less or 5 μm or less, for example, between 1 μm and 5 μm.

The term “number-average size” denotes the dimension given by the statistical particle size distribution to half the population, known as the D50.

Soft-focus filler particles may be of any shape and, for example, may be spherical or non-spherical.

Soft-focus fillers may be of any chemical nature, provided that they are compatible with cosmetic use and do not affect expected properties of soft-focus cosmetic compositions.

Soft-focus fillers may thus be chosen from silica and silicate powders, for example, alumina silicate powders, powders of polymethyl methacrylate (PMMA) type, talc, silica/TiO₂ or silica/zinc oxide composites, polyethylene powders, starch powders, polyamide powders, styrene/acrylic copolymer powders, silicone elastomers and mixtures thereof.

Soft-focus fillers that are particularly suitable for use in embodiments of the invention include but are not limited to talc with a number-average size of less than or equal to 3 microns, for example, talc with a number-average size of 1.8 microns and, for example, TALC P3® by Nippon Talc; NYLON® 12 powder, for example, ORGASOL 2002 EXTRA D NAT COS® by the company Atochem; silica particles surface-treated with a mineral wax 1% to 2% (INCI name: hydrated silica (and) paraffin), such as products sold by the company Degussa; amorphous silica microspheres, such as SUNSPHERE (for example of reference H-53) by the company Asahi Glass; and silica microbeads, such as SB-700® or SB-150® by the company Miyoshi.

In embodiments, soft-focus fillers have a refractive index that is different from that of a medium in which the soft-focus fillers are present.

In embodiments, soft-focus fillers may be substantially colorless.

In embodiments, soft-focus fillers may be present in soft-focus cosmetic compositions in a content ranging from 0.1% to 20% by weight, from 1% to 12% by weight, for example, between 5% and 10%, and, for example, about 8%, relative to the total weight of the composition.

### Coloring Agent

In exemplary embodiments, at least one of soft-focus cosmetic compositions and base compositions may comprise at least one coloring agent.

Thus, soft-focus cosmetic compositions may comprise coloring agents.

In embodiments, compositions may comprise coloring agents when applied directly to naked skin, for example.

In embodiments, soft-focus cosmetic compositions, which are sprayed onto base compositions, may contain, where appropriate, any coloring agent provided that desired H and Th values are not affected.

As used herein, the term “coloring agent” denotes any pigment or dye or mixture of pigments and/or dyes capable of giving soft-focus cosmetic compositions sufficient color to allow the required saturation C*, while at the same time giving desired haze index H and hemispheric transmission factor Th values.

Coloring agents may also be chosen from dyes, for example, water-soluble or liposoluble dyes, or other dyestuffs, provided that incorporation into soft-focus cosmetic compositions does not prevent the desired haze index H and hemispheric transmission factor Th from being obtained. Coloring agent, in embodiments, may thus comprise a water-soluble dye, for instance, caramel.

In embodiments, coloring agents may have a color corresponding to that of the skin onto which soft-focus compositions are intended to be applied, for example, comprising at least one brown, yellow or red pigment.

In embodiments, coloring agents may be free of nacres.

Coloring agents in embodiments may be chosen from any conventional dyestuff used.

Coloring agents may, in embodiments, comprise or consist of particles of at least one pigment. Pigment particles may have undergone, where appropriate, treatments to increase stability of color and to facilitate incorporation into soft-focus cosmetic compositions; pigment particles treated to impart hydrophobicity will be more readily dispersible in an oily phase, for example.

Pigment particles of various embodiments may have various shapes, for example, a substantially spherical or flattened shapes.

In embodiments, pigment particles may have multilayer structures, and for example, transparent cores, such as, for example, cores made of silica.

In embodiments, pigment particles with transparent cores can prevent soft-focus cosmetic compositions from being excessively opacified, which is favorable for obtaining desired H and Th values.

In embodiments, pigments may be non-interference pigments, and may be non-fluorescent.

In embodiments, pigment particles having relatively transparent multilayer structures are included in soft-focus cosmetic compositions and the proportion of pigment may be relatively high, for example, 1% or more by weight, between 1.5% and 10% by weight and between 2% and 8% by weight relative to the total weight of the composition. The proportion may be, for example, between 2% and 5%, for example, about 3%.

Greater amounts of pigments having higher transparency may, in principle, be incorporated into soft-focus cosmetic compositions without causing loss of desired soft focus and hemispheric transmission factor.

Pigment transparency may be quantified by its contrast ratio, which is defined below, this ratio being, in embodiments, for example, between 15 and 65 and 50 or less. The contrast ratio may be 45 or less, 40 or less, or 35 or less.

Coloring agents may comprise pigments having, for example, sericite/brown iron oxide/titanium dioxide/silica type structures. Such pigments are sold, for example, under the reference COVERLEAF NS or JS by the company Chemicals and Catalysts, and has a contrast ratio in the region of 30.

Coloring agents may also comprise pigments having, for example, silica microsphere type structures containing iron oxide. An example of a pigment having this structure and a relatively low contrast ratio is PC BALL PC-LL-100 P by the company Miyoshi, which consists of silica microspheres containing yellow iron oxide.

Relatively low contrast ratio values, combined with haze index and hemispheric transmission factor values, provide satisfactory results, in terms of joint supply of color and coverage, possible and at the same time maintain high soft focus, allowing imperfections to be masked.

In embodiments in which soft-focus cosmetic compositions include mixtures of pigments, proportions of pigments may be adjusted as a function of contrast ratio.

In embodiments, soft-focus cosmetic compositions may be free of pigments with a contrast ratio of greater than 60.

Soft-focus cosmetic compositions may also comprise particles of at least one pigment that is relatively opaque, in particular which has a contrast ratio of greater than 60, with the proviso that the amount introduced into the compositions does not prevent desired H and Th values from being obtained.

Soft-focus cosmetic compositions may thus comprise, for example, conventional pigments having a relatively high contrast ratio, for example, 40 or more, for example pigments of the iron oxide and/or titanium dioxide type.

Soft-focus cosmetic compositions of embodiments of the invention comprise, for example, less than 2% by weight, less than 1.5% by weight, less than 1% by weight and less than 0.75% by weight, for example, between 0.25% and 0.75% by weight or about 0.5% by weight, of such pigments, relative to the total weight of the composition.

### Measurement of the Contrast Ratio

Contrast ratio as used herein is defined in WO 98/52534, the disclosures of which are incorporated herein in their entirety.

Contrast ratio is calculated as follows.

A mixture formed from 5% by weight, relative to the total weight, of the coloring agent to be studied and, for the remainder, from the reference emulsion below is applied to a black opaque support and to a white opaque support, as a film 50 μm thick. This film is dried for 24 hours at 25° C.±1° C. under a pressure of 1 atmosphere.

The reference emulsion has the formulation shown in Table 1.

TABLE 1

| | % by weight |
|---|---|
| Water | 45.83 |
| Methyl p-hydroxybenzoate | 0.45 |
| Chlorphenesin | 0.34 |
| Disodium EDTA | 0.11 |
| Glycerol | 5.62 |
| PEG-8 | 2.25 |
| PEG-20 | 1.12 |
| Magnesium aluminum silicate | 0.9 |
| Sodium lauroyl sarcosinate | 1.68 |
| Titanium dioxide (and) alumina (and) glycerol (and) silica | 3.37 |
| Triethanolamine | 1.35 |
| Stearic acid | 2.7 |
| Glyceryl stearate | 2.02 |
| Butyl p-hydroxbenzoate | 0.17 |
| Isononyl isononanoate | 8.99 |
| Cyclohexasiloxane | 6.57 |
| Dimethicone | 10.28 |
| BIS-PEG-15 methyl ether dimethicone | 2.25 |
| Talc | 1.12 |
| Kaolin | 1.12 |
| Polymethyl methacrylate | 1.69 |

Using a colorimeter, for example a Minolta CR-200 machine, in illuminant D65 mode with a viewing angle of 0°, the tristimulus value Y of the composition is measured at three different points on the black support and at three different points on the white support.

The contrast ratio corresponds to the mean of the three Y values measured on the black support, divided by the mean of the three Y values measured on the white support, and multiplied by 100.

The higher and closer to 100% the contrast ratio, the more opaque the coloring agent. The lower the contrast ratio, the more transparent the coloring agent.

### Physiologically Acceptable Medium

Soft-focus cosmetic compositions, in exemplary embodiments of the invention, comprise physiologically acceptable media.

The term “physiologically acceptable medium” denotes non-toxic mediums that may be applied to skin, for example, the human face and/or neck, lips or nails.

In embodiments, physiologically acceptable media may comprise, where appropriate, liquid fatty phases, which may comprise at least one oil chosen from volatile oils and non-volatile oils, and mixtures thereof.

### Volatile Oil

In exemplary embodiments, the soft-focus cosmetic compositions may comprise liquid fatty phases that may comprise at least one volatile oil.

The term “volatile oil” means any non-aqueous medium capable of evaporating on contact with the skin in less than one hour, at room temperature (25° C.) and atmospheric pressure (760 mmHg). Volatile cosmetic oils, which may be used in embodiments, are liquid at room temperature, and, for example, have vapour pressures, measured at room temperature and atmospheric pressure, ranging from 10⁻³ to 300 mmHg (0.266 Pa to 40 000 Pa), from 0.02 to 300 mmHg (2.66 Pa to 40 000 Pa) and ranging from 0.1 to 90 mmHg (13 Pa to 12 000 Pa).

Volatile oils may be hydrocarbon-based oils, silicone oils, optionally comprising alkyl or alkoxy groups that are pendent or at the end of a silicone chain, and fluoro oils, or a mixture thereof.

In particular embodiments, volatile oils may be cosmetic oils chosen from oils with no flash point, oils with flash points ranging from 40° C. to 100° C., and mixtures thereof, in order to facilitate use. In addition, volatile oils of embodiments may have boiling points at atmospheric pressure of less than 220° C., or less than 210° C., and, for example, ranging from 110 to 210° C. These volatile oils may, for example, not be monoalcohols containing at least seven carbon atoms.

In embodiments, volatile oils that may be used include but are not limited to linear or cyclic silicone oils with a viscosity, at room temperature, of less than 8 cSt and, for example, containing from 2 to 7 silicon atoms, these silicones optionally comprising alkyl or alkoxy groups containing from 1 to 10 carbon atoms.

In embodiments, volatile silicone oils that may be used in accordance with embodiments include but are not limited to octamethylcyclotetrasiloxane, decamethyl-cyclopentasiloxane, dodecamethylcyclohexasiloxane, heptamethylhexyltrisiloxane, heptamethyloctyltrisiloxane, hexamethyldisiloxane, octamethyltrisiloxane, decamethyl-tetrasiloxane, dodecamethylpentasiloxane and mixtures thereof.

Other volatile oils that may be used in accordance with embodiments include but are not limited to hydrocarbon-based volatile oils containing from 8 to 16 carbon atoms and mixtures thereof, and branched C₈-C₁₆ alkanes, for instance C₈-C₁₆ isoalkanes (also known as isoparaffins), isododecane, isodecane and isohexadecane, for example, the oils sold under the trade names ISOPAR® or PERMETHYL®, and branched C₈-C₁₆ esters, for instance, isohexyl neopentanoate, and mixtures thereof.

Isododecane (PERMETHYL 99 A®), C₈-C₁₆ isoparaffins, for instance, ISOPAR L, E, G or H®, mixtures thereof, optionally combined with decamethyltetrasiloxane or with cyclopentasiloxane, or mixtures thereof, may be used in embodiments.

Volatile fluoro oils may also be used in embodiments of the invention.

In embodiments, volatile oils may be present in soft-focus cosmetic compositions in a content ranging, for example, from 5% to 97.5% by weight and from 20% to 75% by weight, relative to the total weight of the composition.

### Non-Volatile Oil

In exemplary embodiments, soft-focus cosmetic compositions may also comprise non-volatile fatty fractions. Non-volatile fatty fractions may comprise at least one non-volatile oil.

The term “non-volatile oil” means fatty substances that are liquid at room temperature (20° C.) and that do not evaporate at that temperature.

In embodiments, soft-focus cosmetic compositions include non-volatile oils including but not limited to:

poly(C₁-C₂₀)alkylsiloxanes and, for example, those containing trimethylsilyl end groups, for example, those with a viscosity of less than 0.06 m²/s, among which mention may be made of linear polydimethylsiloxanes and alkylmethylpolysiloxanes such as cetyldimethicone (CTFA name),

silicones modified with optionally fluorinated aliphatic and/or aromatic groups, or with functional groups such as hydroxyl, thiol and/or amine groups,

phenylsilicone oils, for example, phenyl trimethicones,

oils of plant or mineral origin, such as liquid paraffin, liquid petroleum jelly, perhydrosqualene, lanolin, apricot oil, wheatgerm oil, sweet almond oil, beauty-leaf oil, sesame seed oil, macadamia oil, grapeseed oil, rapeseed oil, coconut oil, groundnut oil, sunflower oil, palm oil, castor oil, avocado oil, jojoba oil, olive oil or cereal germ oil and liquid fractions of shea butter; fatty acid esters of polyols, for example, liquid triglycerides, isopropyl myristate, Miglyol oil, isostearyl neopentanoate, 2-ethylhexyl palmitate, castor oil, tributyl acetyl citrate; alcohols, for example, 2-octyldodecanol; acetylglycerides; octanoates, decanoates or ricinoleates of alcohols or of polyalcohols; fatty acid triglycerides; glycerides, and fluoro and perfluoro oils,

amide compounds, for example, those described in patent application WO 98/56754, for instance N-neopentanoyl-2-octyldodecylamine, N-neopentanoyl-2-butyloctylamine, N-(3,5,5-trimethylhexanoyl)-2-octyldodecylamine or N-(3,5,5-trimethylhexanoyl)-2-butyloctylamine, and

mixtures thereof.

In particular embodiments, soft-focus cosmetic compositions may comprise one or more oils chosen from sesame oil, lanolin, isopropyl myristate, Miglyol oil, isostearyl neopentanoate, 2-ethylhexyl palmitate, castor oil, acetyl tributyl citrate, 2-octyldodecanol, N-pentanoyl-2-octyldodecylamine and polydimethylsiloxanes and/or mixtures thereof.

Non-volatile fatty fractions of soft-focus cosmetic compositions in embodiments may also comprise non-liquid fatty phases and, for example, at least one fatty substance chosen from waxes, gums, resins, pasty fatty substances of animal, plant, mineral or synthetic origin and mixtures thereof. In embodiments, the content of non-liquid fatty phases in soft-focus cosmetic compositions may be adjusted for application by spraying.

In embodiments, non-volatile fatty fractions of soft-focus cosmetic compositions may be present in a content, for example ranging from 1% to 85% by weight and from 1% to 30% by weight, relative to the total weight of the composition.

### Aqueous Phase

In exemplary embodiments, soft-focus cosmetic compositions may also comprise aqueous phases. Aqueous phases may consist essentially of water. However, aqueous phases may also comprise mixtures of water and of water-miscible organic solvents (water miscibility of greater than 50% by weight at 25° C.), for instance lower monoalcohols containing from 1 to 5 carbon atoms, such as ethanol and isopropanol, glycols containing from 2 to 8 carbon atoms, such as propylene glycol, ethylene glycol, 1,3-butylene glycol and dipropylene glycol, C₃-C₄ ketones and C₂-C₄ aldehydes.

Aqueous phases (water and optionally water-miscible organic solvents) may be present in soft-focus cosmetic compositions in a content, for example, ranging from 1% to 95% by weight, from 3% to 80% by weight and from 5% to 60% by weight, relative to the total weight of the composition.

### Additional Optional Components

Soft-focus cosmetic compositions of exemplary embodiments may also comprise film-forming polymers, as described below, under conditions that allow spraying.

Soft-focus cosmetic compositions of exemplary embodiments may also contain non-liquid fatty phases.

In exemplary embodiments, soft-focus cosmetic compositions may also contain adjuvants that are common in cosmetics, such as emulsifiers, hydrophilic or lipophilic gelling agents, hydrophilic or lipophilic active agents, preserving agents, antioxidants, solvents, fragrances, physical sunblocks and chemical sunscreens, odour absorbers and pH regulators. Examples of such adjuvants are described below.

Soft-focus cosmetic compositions of exemplary embodiments may also contain surfactants, for example, those mentioned below with respect to base compositions.

In exemplary embodiments, fluid consistency of soft-focus cosmetic compositions may vary according to spraying devices used and according to propellants used, in embodiments including aerosol type spraying devices.

In embodiments, soft-focus cosmetic compositions may be in emulsion form, for example obtained by dispersing fatty phases in aqueous phases (0/W) or conversely (W/O), triple emulsions (W/O/W or O/W/O), vesicular dispersions of ionic (liposomes or oleosomes) and/or nonionic type (niosomes) and/or dispersions of nanocapsules or nanospheres.

In embodiments in which soft-focus cosmetic compositions are in emulsion form, the proportion of fatty phases in soft focus cosmetic compositions may range, for example, from 5% to 80% by weight and from 5% to 50% by weight, relative to the total weight of the composition.

Soft-focus cosmetic compositions may also be in the form of sprayable gels, for example, thixotropic gels or gels that are soluble in propellent gas, for example.

### Base Composition

In exemplary embodiments, base compositions may also be applied. Base compositions according to such embodiments may also comprise physiologically acceptable media.

In embodiments, physiologically acceptable media are generally adapted to surfaces onto which base compositions are to be applied and also may also be adapted to intended packaging.

Thus, base compositions may be more or less fluid and may have the appearance of colored or uncolored creams, ointments, milks, lotions, serums, pastes, mousses or powders. Base compositions may also be in solid form, such as stick or dish form, or may have been deposited by coating onto paper.

Base compositions may be in anhydrous forms, emulsion forms or gel forms.

Base compositions may be in any galenical form normally used in cosmetics, and may, for example, be in the form of optionally gelled oily solutions, emulsions obtained by dispersing fatty phases in aqueous phases (O/W) or conversely (W/O), triple emulsions (W/O/W or O/W/O), aqueous gels, vesicular dispersions of ionic type (liposomes or oleosomes) and/or nonionic type (niosomes) and/or dispersions of nanocapsules or nanospheres.

In embodiments in which base compositions are in emulsions, the proportion of fatty phase under consideration may range, for example, from 5% to 80% by weight and for example, from 5% to 50% by weight, relative to the total weight of the composition.

### Aqueous Phase

Base compositions of exemplary embodiments may comprise at least one aqueous phase.

In embodiments, aqueous phases may consist essentially of water. However, aqueous phases may also comprise mixtures of water and of water-miscible organic solvents (miscible in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

In embodiments, aqueous phases (water and optionally water-miscible organic solvents) may be present in base compositions in a content, for example, ranging from 1% to 95% by weight, from 3% to 80% by weight, and from 5% to 60% by weight, relative to the total weight of the base composition.

### Fatty Phase

Base compositions of exemplary embodiments may also comprise a fatty phase.

In particular, fatty phases of base compositions of embodiments may comprise at least one oil including but not limited to:

hydrocarbon-based oils of animal origin, such as perhydrosqualene;

hydrocarbon-based oils of plant origin, such as liquid triglycerides of fatty acids containing from 4 to 10 carbon atoms and the liquid fraction of karite butter;

synthetic esters and synthetic ethers, for example, of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as parleam oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils, for instance those described in document JP-A-2 295 912;

silicone oils, for instance volatile or non-volatile polymethylsiloxanes (PDMSs) containing a linear or cyclic silicone chain, that are liquid or pasty at room temperature, for example, cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltri-methylsiloxydiphenylsiloxanes, diphenyldimethicones, diphenylmethyldiphenyltrisiloxanes, 2-phenylethyltrimethylsiloxysilicates and polymethylphenylsiloxanes; and

mixtures thereof.

### Waxes

In exemplary embodiments, base compositions may also comprise at least one wax, at least one gum and/or at least one pasty fatty substance, of plant, animal, mineral or synthetic origin, of silicone or non-silicone nature.

Waxes may be hydrocarbon-based waxes, silicone waxes and/or fluoro waxes, optionally comprising ester or hydroxyl functions. They are, for example, of natural origin.

In embodiments, waxes may represent from 0.01% to 10% by weight and for example, from 0.1% to 5% by weight relative to the total weight of the base composition.

According to exemplary embodiments, base compositions may be wax-free.

### Film Forming Polymer

Base compositions may also comprise, in various exemplary embodiments, at least one film-forming polymer.

According to the present invention, the term “film-forming polymer” means a polymer that is capable of forming, by itself or in the presence of an auxiliary film-forming agent, a continuous film that adheres to the skin.

In embodiments, film-forming polymers capable of forming a hydrophobic film, i.e., polymers whose films have a solubility in water at 25° C. of less than 1% by weight, may be used.

In embodiments, film-forming polymers may be at least one polymer chosen from the group comprising:

water-soluble film-forming polymers,

aqueous dispersions of water-dispersible film-forming polymer particles, also known as “latices”: in this case, the composition of foundation type must comprise an aqueous phase,

liposoluble film-forming polymers,

lipodispersible film-forming polymers in the form of non-aqueous dispersions of polymer particles, preferably dispersions of polymer particles surface-stabilized, where appropriate, with at least one stabilizer, in one or more silicone oils and/or hydrocarbon-based oils; these non-aqueous dispersions are also known as “NADs”, and

mixtures thereof.

Aqueous dispersions of film-forming polymers that may be used in embodiments include but are not limited to acrylic dispersions sold under the names NEOCRYL XK-90®, NEOCRYL A-1070®, NEOCRYL A-1090®, NEOCRYL BT-62®, NEOCRYL A-1079® and NEOCRYL A-523® by the company Avecia-Neoresins, DOW LATEX 432® by the company Dow Chemical, DAITOSOL 5000 AD® by the company Daito Kasey Kogyo; or aqueous dispersions of polyurethane sold under the names NEOREZ R-981® and NEOREZ R-974® by the company Avecia-Neoresins, AVALURE UR-405®, AVALURE UR-410®, AVALURE UR-425®, AVALURE UR-450®, SANCURE 875®, SANCURE 861®, SANCURE 878® and SANCURE 2060® by the company Goodrich, IMPRANIL 85® by the company Bayer and AQUAMERE H-1511® by the company Hydromer.

Aqueous dispersions of film-forming polymers that may also be used in embodiments include but are not limited to polymer dispersions resulting from free-radical polymerization of one or more free-radical monomers inside and/or partially at surfaces of pre-existing particles of at least one polymer chosen from the group consisting of polyurethanes, polyureas, polyesters, polyesteramides and/or alkyd polymers. These polymers are generally referred to as hybrid polymers.

Examples of water-soluble film-forming polymers that may be used in embodiments include but are not limited to proteins, for instance proteins of plant origin, anionic, cationic, amphoteric or nonionic chitin or chitosan polymers, cellulose polymers, acrylic polymers or copolymers, vinyl polymers and polymers of natural origin, which are optionally modified, and mixtures thereof.

Exemplary dispersions of film-forming polymers in a liquid fatty phase, in the presence of stabilizers, are described in EP-A-749 746, EP-A-923 928 and EP-A-930 060, the content of which is incorporated herein by reference.

Non-limiting examples of liposoluble polymers that may be used in embodiments include but are not limited to copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

In embodiments, film-forming polymers may be present in compositions in solids contents ranging from 0.01% to 20% by weight and from 0.5% to 10% by weight relative to the total weight of the composition.

### Adjuvants

In exemplary embodiments, base compositions may also comprise adjuvants that are common in cosmetics, such as emulsifiers, hydrophilic or lipophilic gelling agents, dyestuffs, hydrophilic or lipophilic active agents, preserving agents, antioxidants, solvents, fragrances, fillers, physical sunblocks and chemical sunscreens, odor absorbers and pH regulators.

The amounts of various adjuvants in embodiments may be those conventionally used in the cosmetic arts, for example from 0.01% to 20% of the total weight of the base composition. Depending on their nature, adjuvants may be introduced into fatty phases or into aqueous phases.

In any case, adjuvants, and proportions thereof, will be chosen so as not to harm desired properties according to embodiments of the invention.

### Surfactant

Base compositions may also comprise, in exemplary embodiments, at least one surfactant.

Surfactants may be chosen from anionic and nonionic surfactants. Reference may be made to the “Encyclopedia of Chemical Technology, Kirk-Othmer”, volume 22, pp. 333-432, 3rd edition, 1979, Wiley, for the definition of the properties and functions of surfactants, in particular pp. 347-377 of this reference, for the anionic and nonionic surfactants.

In embodiments, base compositions may include surfactants including but are not limited to:

nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,

anionic surfactants: C₁₆-C₃₀ fatty acids neutralized with amines, aqueous ammonia or alkaline salts, and mixtures thereof.

In embodiments, emulsifiers and co-emulsifiers may be used, including but not limited to O/W emulsifiers such as fatty acid esters of polyethylene glycol, for example, PEG-100 stearate, and fatty acid esters of glycerol, such as glyceryl stearate, and also W/O emulsifiers available, for example, under the trade names ABIL WE09, ABIL EM 90, ABIL EM97 from the company Degussa Goldschmidt, or the mixture of acetyl ethylene glycol stearate and of glyceryl tristearate sold by the company Guardian under the trade name UNITWIX.

Hydrophilic gelling agents that may be used in embodiments include but are not limited to carboxyvinyl polymers (carbomer), acrylic copolymers, such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

### Coloring Agent

As dyestuffs that may be used in base compositions of exemplary embodiments, mention may be made of lipophilic dyes, hydrophilic dyes, pigments and nacres usually used in cosmetic or dermatological compositions and mixtures thereof.

Liposoluble dyes may include, for example, Sudan Red, DC Red 17, DC Green 6, n-carotene, soybean oil, Sudan Brown, DC Yellow 11, DC Violet 2, DC Orange 5 and quinoline yellow.

Pigments that may be used in embodiments may be white or colored, mineral and/or organic, and coated or uncoated. Among mineral pigments that may be mentioned are titanium dioxide, optionally surface-treated, zirconium oxide or cerium oxide, and also iron oxide or chromium oxide, manganese violet, ultramarine blue, chromium hydrate and ferric blue. Among organic pigments that may be mentioned are carbon black, pigments of D&C type and lakes based on cochineal carmine or on barium, strontium, calcium or aluminium.

In embodiments, nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and nacreous pigments based on bismuth oxychloride.

In embodiments, pigments may have undergone surface treatments.

Among pigments that may be used in base compositions in embodiments, mention may also be made of goniochromatic pigments.

### Fillers

In exemplary embodiments, fillers that may be used in base compositions include but are not limited to: fillers described above in relation to soft-focus cosmetic compositions, pigments, starch crosslinked with octenylsuccinic anhydride sold by the company National Starch under the name DRY FLO PLUS (28-1160); polyamide particles and, for example, those sold under the name ORGASOL by the company Atochem; microspheres based on acrylic copolymers, such as those made of ethylene glycol dimethacrylate/lauryl methacrylate copolymer sold by the company Dow Corning under the name POLYTRAP; expanded powders such as hollow microspheres and, for example, the microspheres sold under the name EXPANCEL by the company Kemanord Plast or under the name MICROPEARL F 80 ED by the company Matsumoto; polymethacrylate-type powders (PMMA), silicone elastomers and silica powders of the SUNSPHERE type, silicone resin microbeads such as those sold under the name TOSPEARL by the company Toshiba Silicone; and mixtures thereof.

In embodiments, fillers may be present in amounts ranging from 0.01% to 20% by weight and from 1% to 10% by weight, relative to the total weight of the base composition.

In embodiments, fillers such as silica, mica and titanium dioxide may be used.

### Active Agents

In exemplary embodiments, active agents that are common in cosmetics or dermatology may be used in base compositions and/or soft-focus cosmetic compositions. Active agents include but are not limited to any active agent known for its activity on ageing of skin, for instance keratolytic agents or prodesquamating agents, for example α-hydroxy acids, β-hydroxy acids, α-keto acids, β-keto acids, retinoids and esters thereof, retinal, and retinoic acid and its derivatives; venotonic plant extracts such as extracts of ruscus and/or of common horse chestnut; xanthine bases such as caffeine; vitamins, for instance vitamins A, B3, PP, B5, E, K1 and/or C and derivatives of these vitamins and esters thereof; free-radical scavengers; sunscreens; moisturizers, for instance polyols; ceramides; DHEA and its derivatives; coenzyme Q10; bleaching agents and depigmenting agents, for instance kojic acid, extracts of skullcap, of mulberry, of liquorice and/or of camomile; para-aminophenol derivatives, arbutin and derivatives thereof, and mixtures thereof.

For use in cosmetic treatment of greasy or combination skin, exemplary embodiments may comprise base compositions and/or soft-focus cosmetic compositions containing at least one active agent chosen from: vitamins B3 and B5; zinc salts, in particular zinc oxide and zinc gluconate; salicylic acid and its derivatives, such as 5-n-octanoylsalicylic acid; triclosan; capryloylglycine; an extract of clove; octopirox; hexamidine; and azelaic acid and its derivatives.

In embodiments, UVA and/or UVB screening agents chosen from organic screening agents and mineral screening agents, optionally including hydrophobic coatings may also be introduced into base compositions and/or into soft-focus cosmetic compositions.

### Makeup Kit

Exemplary embodiments also include makeup kits. In embodiments, makeup kits comprise at least one base composition and at least one soft-focus cosmetic composition, which is different from the base compositions.

In embodiments, makeup kits may also comprise means for applying base compositions to skin, for example.

In embodiments, base compositions and soft-focus cosmetic compositions, forming makeup kits, may or may not be packaged in separate packaging articles.

This invention is illustrated by the following Examples, which are merely for the purpose of illustration.

### EXAMPLES

### Example 1

### Base Composition Consisting of a Foundation

A base composition according to embodiments of the invention is prepared using the formulation set forth in Table 2.

TABLE 2

| | % by |
|---|---|
| | weight |
| 1,3-Butylene glycol | 10.00 |
| Hectorite modified with distearyldimethylammonium chloride | 1.60 |
| (sold under the name BENTONE 38V by Elementis) | |
| Preserving agents | 0.90 |
| Cyclopentadimethylsiloxane | 11.36 |
| Isostearyl neopentanoate | 0.50 |
| Sodium Chloride | 0.70 |
| Isododecane | 13.00 |
| Cyclohexadimethylsiloxane | 8.00 |
| Polydimethylsiloxane (DC 200 FLUID 5 cSt sold by Dow | 2.00 |
| Corning) | |
| Cetyl dimethicone copolyol (sold under the name ABIL | 0.80 |
| EM 90 by Goldschmidt) | |
| Polyglyceryl isostearate | 0.60 |
| Isoeicosane | 2.00 |
| Hexyl laurate | 0.60 |
| Hollow microspheres of polymethyl methacrylate (sold under | 2.00 |
| the name COVABEAD LH 85 by Wackherr) | |
| Polymethyl methacrylate powder (sold under the name | 2.00 |
| JURYMER MB1 by Nihon Junyaku) | |
| Oxyethylenated polydimethylsiloxane (sold under the name | 4.48 |
| KF-6017 from Shin-Etsu) | |
| Nacre | 1.00 |
| Yellow iron oxide coated with perfluoroalkyl phosphate as a | 2.40 |
| dispersion in decamethylcyclopentasiloxane/dimethicone | |
| copolyol (sold under the name FA50DYF by Kobo) | |
| Brown iron oxide coated with perfluoroalkyl phosphate as a | 0.93 |
| dispersion in cyclomethicone/dimethyl polysiloxane copolyol | |
| (sold under the name FA50DRF by Kobo) | |
| Black iron oxide coated with perfluoroalkyl phosphate as a | 0.44 |
| dispersion in decamethylcyclopentasiloxane/dimethicone | |
| copolyol (sold under the name FA65DBF by Kobo) | |
| Alumina-treated titanium oxide coated with perfluoroalkyl | 9.54 |
| phosphate in decamethylcyclopentasiloxane/dimethicone | |
| copolyol (sold under the name FA65DF by Kobo) | |
| water | qs 100.00 |

Uncolored Soft-Focus Cosmetic Composition Intended to be Sprayed onto the Above Base Composition:

An uncoloured soft-focus cosmetic composition according to embodiments is prepared using the formulation set forth in Table 3.

TABLE 3

| | % by weight |
|---|---|
| Polyglyceryl-4 isostearate (and) hexyl laurate (and) cetyl | 9.00 |
| PEG/PPG-10/1 dimethicone | |
| Acetylated glycol stearate | 0.70 |
| Butyl p-hydroxybenzoate | 0.15 |
| 2-Oleamido-1,3-octadecanediol | 0.04 |
| Cyclopentasiloxane (and) disteardimonium hectorite(and) | 8.00 |
| denatured alcohol | |
| Cyclopentasiloxane | 14.25 |
| Dimethicone | 4.00 |
| Isododecane | 2.60 |
| Talc P3 from the company Nippon Talc | 8.00 |
| Water | 47.06 |
| Magnesium sulfate | 0.70 |
| Methyl p-hydroxybenzoate | 0.25 |
| Propylene glycol | 5.00 |
| Chlorphenesin | 0.25 |

The uncolored soft-focus composition is applied onto the base composition using a spraying device of aerographic type.

A substantial reduction in the perception of the skin imperfections may be observed.

### Example 2

### Colored Soft-Focus Cosmetic Composition that May be Sprayed onto Naked Skin

A colored soft-focus cosmetic composition according to embodiments of the invention is prepared using the formulation set forth in Table 4.

TABLE 4

| | % by weight |
|---|---|
| Polyglyceryl-4 isostearate (and) hexyl laurate (and) cetyl | 9.00 |
| PEG/PPG-10/1 dimethicone | |
| Acetylated glycol stearate | 0.70 |
| Butyl p-hydroxybenzoate | 0.15 |
| 2-Oleamido-1,3-octadecanediol | 0.04 |
| Cyclopentasiloxane (and) disteardimonium hectorite(and) | 8.00 |
| denatured alcohol | |
| Cyclopentasiloxane | 14.25 |
| Dimethicone | 4.00 |
| Isododecane | 2.60 |
| Coverleaf NS pigment from the company Chemicals and | 3.00 |
| Catalysts | |
| Talc P3 from the company Nippon Talc | 8.00 |
| Water | 44.06 |
| Magnesium sulfate | 0.70 |
| Methyl p-hydroxybenzoate | 0.25 |
| Propylene glycol | 5.00 |
| Chlorphenesin | 0.25 |

The composition is applied using a spraying device of aerographic type.

A supply of color and a reduction in the perception of the relief defects may be observed.

### Example 3

### Colored Soft-Focus Cosmetic Composition that May be Sprayed onto Naked Skin

A colored soft-focus cosmetic composition according to embodiments of the invention is prepared using the formulation set forth in Table 5.

TABLE 5

| | % by weight |
|---|---|
| Polyglyceryl-4 isostearate (and) hexyl laurate (and) cetyl | 9.00 |
| PEG/PPG-10/1 dimethicone | |
| Acetylated glycol stearate | 0.702 |
| Butyl p-hydroxybenzoate | 0.15 |
| 2-Oleamido-1,3-octadecanediol | 0.04 |
| Cyclopentasiloxane (and) disteardimonium hectorite(and) | 8.00 |
| denatured alcohol | |
| Cyclopentasiloxane | 14.25 |
| Dimethicone | 4.00 |
| Isododecane | 2.60 |
| Iron oxides (and) disodium stearoyl glutamate (and) | 0.18 |
| aluminium hydroxide | |
| Titanium dioxide (and) disodium stearoyl glutamate (and) | 0.32 |
| aluminium hydroxide | |
| Talc P3 from the company Nippon Talc | 8.00 |
| Water | 46.56 |
| Magnesium sulfate | 0.70 |
| Methyl p-hydroxybenzoate | 0.25 |
| Propylene glycol | 5.00 |
| Chlorphenesin | 0.25 |

The composition is applied as previously using a spraying device of aerographic type. A reduction in the visibility of the color imperfections and relief imperfections of the skin is again observed.

Throughout the description, including the claims, the term “comprising a” should be understood as being synonymous with “comprising at least one”, unless otherwise mentioned.

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

What is claimed is:

1. A cosmetic treatment process comprising:
applying a soft-focus cosmetic composition to a surface of skin, mucous membrane or nails by spraying,
wherein the soft-focus cosmetic composition comprises at least one filler in a physiologically acceptable medium, the at least one filler giving the soft-focus cosmetic composition a haze index H of 40% or more and a hemispheric transmission factor Th of 70% or more.

2. The cosmetic treatment process according to claim 1, wherein the soft-focus cosmetic composition has a haze index H of 45% or more.

3. The cosmetic treatment process according to claim 1, wherein the soft-focus cosmetic composition has a haze index H of 50% or more.

4. The cosmetic treatment process according to claim 1, wherein the soft-focus cosmetic composition has a haze index H of 60% or more.

5. The cosmetic treatment process according to claim 1, wherein the soft-focus cosmetic composition has a haze index H of 70% or more.

6. The cosmetic treatment process according to claim 1, wherein the soft-focus cosmetic composition has a hemispheric transmission factor Th of 75% or more.

7. The cosmetic treatment process according to claim 1, wherein the soft-focus cosmetic composition has a hemispheric transmission factor Th of 80% or more.

8. The cosmetic treatment process according to claim 1, wherein the soft-focus cosmetic composition has a hemispheric transmission factor Th of 85% or more.

9. The cosmetic treatment process according to claim 1, wherein the filler comprises particles with a number-average size of 15 μm or less.

10. The cosmetic treatment process according to claim 1, wherein the filler comprises particles with a number-average size of 10 μm or less.

11. The cosmetic treatment process according to claim 1, wherein the filler comprises particles with a number-average size of 7.5 μm or less.

12. The cosmetic treatment process according to claim 1, wherein the filler comprises particles with a number-average size of 5 μm or less.

13. The cosmetic treatment process according to claim 1, wherein the filler consists of particles with a number-average size of 15 μm or less.

14. The cosmetic treatment process according to claim 1, wherein the filler consists of particles with a number-average size of 10 μm or less.

15. The cosmetic treatment process according to claim 1, wherein the filler consists of particles with a number-average size of 7.5 μm or less.

16. The cosmetic treatment process according to claim 1, wherein the filler consists of particles with a number-average size of 5 μm or less.

17. The cosmetic treatment process according to claim 1, wherein the filler is at least one filler selected from the group consisting of silica powders, silicate powders, talc, powders of polymethyl methacrylate (PMMA) type, silica/TiO₂ composites, silica/zinc oxide composites, polyethylene powders, starch powders, polyamide powders, styrene/acrylic copolymer powders, silicone elastomers and mixtures thereof.

18. The cosmetic treatment process according to claim 1, wherein the filler is selected from the group consisting of alumina silicate powders.

19. The cosmetic treatment process according to claim 1, wherein the filler is selected from the group consisting of talc particles with an average size of 3 μm or less, Nylon 12 powder, silica particles surface-treated with a mineral wax, amorphous silica microspheres, silica microbeads and mixtures thereof.

20. The cosmetic treatment process according to claim 1, wherein the filler is present in a proportion of from 0.1% to 20% by weight relative to a total weight of the soft-focus cosmetic composition.

21. The cosmetic treatment process according to claim 1, wherein the filler is present in a proportion of from 1% to 12% by weight relative to a total weight of the soft-focus cosmetic composition.

22. The cosmetic treatment process according to claim 1, wherein the filler is present in a proportion of from 5% to 10% by weight relative to a total weight of the soft-focus cosmetic composition.

23. The cosmetic treatment process according to claim 1, wherein:
the soft-focus cosmetic composition further comprises at least one liquid fatty phase; and the liquid fatty phase comprises at least one oil selected from the group consisting of volatile oils, non-volatile oils and mixtures thereof.

24. The cosmetic treatment process according to claim 23, wherein the liquid fatty phase comprises at least one volatile oil selected from the group consisting of linear silicone oils with a viscosity at room temperature of less than 8 cSt, cyclic silicone oils with a viscosity at room temperature of less than 8 cSt, volatile hydrocarbon-based oils containing from 8 to 16 carbon atoms, fluoro oils and mixtures thereof.

25. The cosmetic treatment process according to claim 23, wherein the liquid fatty phase comprises at least one volatile oil selected from the group consisting of isododecane, C₈ to C₁₆ isoparaffins and mixtures thereof.

26. The cosmetic treatment process according to claim 25, wherein the liquid fatty phase comprises at least one volatile oil and at least one member selected from the group consisting of decamethyltetrasiloxane, cyclopentasiloxane and mixtures thereof.

27. The cosmetic treatment process according to claim 23, wherein the liquid fatty phase comprises at least one volatile oil in a content ranging from 5% to 97.5% by weight relative to the total weight of the soft-focus cosmetic composition.

28. The cosmetic treatment process according to claim 23, wherein the liquid fatty phase comprises at least one volatile oil in a content ranging from 20% to 75% by weight relative to the total weight of the soft-focus cosmetic composition.

29. The cosmetic treatment process according to claim 23, wherein the liquid fatty phase comprises at least one non-volatile oil selected from the group consisting of poly(C₁-C₂₀)alkylsiloxanes, silicones modified with aliphatic groups, silicones modified with aromatic groups, phenylsilicone oils, oils of plant origin, oils of mineral origin, amide compounds and mixtures thereof.

30. The cosmetic treatment process according to claim 29, wherein the non-volatile oil is selected from the group consisting of sesame oil, lanolin, isopropyl myristate, Miglyol oil, isostearyl neopentanoate, 2-ethylhexyl palmitate, castor oil, acetyl tributyl citrate, 2-octyldodecanol, N-pentanoyl-2-octyldodecylamine, polydimethylsiloxanes and mixtures thereof.

31. The cosmetic treatment process according to claim 1, wherein the soft-focus cosmetic composition further comprises a non-liquid fatty phase.

32. The cosmetic treatment process according to claim 1, wherein the soft-focus cosmetic composition further comprises a non-volatile fatty fraction present in a proportion of from 1% to 85% by weight relative to a total weight of the soft-focus cosmetic composition.

33. The cosmetic treatment process according to claim 1, wherein the soft-focus cosmetic composition comprises a non-volatile fatty fraction present in a proportion of from 1% to 30% by weight relative to a total weight of the soft-focus cosmetic composition.

34. The cosmetic treatment process according to claim 1, further comprising applying at least one base composition to the surface, wherein:
the base composition is different from the soft-focus cosmetic composition; and
applying the base composition is performed before applying the soft-focus cosmetic composition.

35. The cosmetic treatment process according to claim 34, wherein the base composition is colored.

36. The cosmetic treatment process according to claim 1, further comprising applying at least one base composition to the surface by spraying, wherein:
the base composition is different from the soft-focus cosmetic composition; and
applying of the soft-focus cosmetic composition is performed simultaneously with applying of the base composition.

37. The cosmetic treatment process according to claim 34, wherein at least one of the soft-focus cosmetic composition and the base composition comprises at least one coloring agent.

38. The cosmetic treatment process according to claim 36, wherein at least one of the soft-focus cosmetic composition and the base composition comprises at least one coloring agent.
